(19) United States
(12) Patent Application Publication
(10) Pub. No.: US 2021/0403972 A1
(43) Pub. Date: Dec. 30, 2021

(54) Novel Methods

(71) Applicant: Colgate-Palmolive Company, New York, NY (US)

(72) Inventors: Tulika SARMA, Hillsborough, NJ (US); Kristina FABIJANIC, Jersey City, NJ (US)

(73) Assignee: Colgate-Palmolive Company, New York, NY (US)

(21) Appl. No.: 17/358,945

(22) Filed: Jun. 25, 2021

Related U.S. Application Data
(60) Provisional application No. 63/044,518, filed on Jun. 26, 2020.

(51) Int. Cl. C12Q 1/04; G02B 21/00; G01Q 60/24; G01N 21/64

(52) U.S. Cl. CPC C12Q 1/04; G02B 21/0076; A61C 7/08; G01N 21/6402; G01Q 60/24

(57) ABSTRACT

This invention is directed to methods of detecting biofilm treated in situ with an active ingredient, and subsequently imaging or detecting types or varieties of biofilm and/or architectural changes in biofilm when treated with certain actives. Additionally, the invention contemplates screening assays to discover further candidate compounds that can affect biofilm growth and formation, as it relates to the maintenance of oral health and prevention of oral diseases.

### FIELD OF THE INVENTION

This invention in one aspect is directed to methods of detecting biofilm. Additionally, the invention contemplates screening assays to discover further candidate compounds that can affect biofilm growth and formation.

### BACKGROUND OF THE INVENTION

The oral biofilm comprises numerous types of microbes, mainly bacteria, that colonize oral surfaces, which can include dental enamel and oral mucosa. In general, good oral health is associated with systemic health. Therefore, the type of bacteria or biofilm found in the oral cavity can offer a great deal of information both about both the local health in the oral cavity, but also information generally about the systemic health of an individual.

Researchers have been evaluating the complex nature of oral biofilms for quite some time. Oral microbial biofilms, generally speaking, are three-dimensional structured bacterial communities attached to a solid surface like the enamel of the teeth, or the surface of the root or dental implants, and are embedded in an exo-polysaccharide matrix. Oral biofilms can provide information about the health of an individual.

Because of the importance of researching and characterizing oral biofilms, this has led to the development of a number of methods and assays for their evaluation. One common or conventional method has been to culture various different oral biofilms and study them in culture. Although, this particular approach is sometimes problematic as certain estimates indicate that many oral microbiota may be unculturable. Thus, methods of culturing biofilms in laboratory cell cultures may not be adequately representative of the actual environment that exists in the oral cavity.

There are advantages and disadvantages to studying plaque or biofilm that is grown in vitro—e.g., in a laboratory cell culture—and in vivo—e.g., samples which are taken directly from a subject's oral cavity. In vitro methods can allow researchers to study the biofilm growth under standardized and simplified conditions. The drawback to such methods being that in vitro methods may not completely replicate the actual environment of the oral cavity. On the other hand, in vivo experiments have the advantage that they mimic natural oral conditions. The disadvantage to in vivo experiments is that they can be more complex and the conditions can be less controlled. Thus, while in vivo experiments may be more representative of the oral care environment, they present hurdles given the difficulties attendant in running such experiments.

However, given that the study of biofilm and microflora, within the oral cavity, is critical to understanding the many various disease processes affecting oral health, developing in vivo experiments is paramount for researchers. Moreover, understanding plaque biofilm architecture and functionality in nature, can directly relate to understanding which active molecules, and delivery designs, are effective for plaque biofilm control. Thus, there is a need for a detection method which can accurately characterize, detect, and determine the biofilm which naturally occurs in the oral cavity.

### SUMMARY OF THE INVENTION

The methods contemplated by the present invention utilize the surprising discovery that under certain conditions that toothpastes containing metal salts (e.g., stannous fluoride) can alter the architecture, structure, and types of biofilm that grow in situ in the oral cavity. In one aspect, it is surprisingly found that biofilm subjected to stannous fluoride toothpaste does not grow as thickly, relative to control standards. Moreover, it was found that the volume of this biofilm is also different from samples treated with a control. Without being bound by theory, the use of toothpastes containing stannous salts are also believed to alter the types of species and strains of biofilm that grow in situ, relative to a control.

In one aspect, the Applicant's detection method demonstrates that upon continuous brushing with an oral composition, e.g., a toothpaste, comprising stannous fluoride (an active), that the bacterial colonization after brushing teeth becomes less densely populated on the dental surface. The pattern and the population growth of the microcolonies and their spatial structure differs from the pattern of growth when brushing with a non-stannous fluoride toothpaste. The size of bacterial colonies are much smaller and not evenly distributed and there is a reduction in stickiness (e.g., the adhesion or adhesive forces of the colonies to the hard surface) and an increase in stiffness of these bacterial colonies. In still another aspect, the present invention contemplates a screening method that can be used to identify more efficacious oral care compositions and actives. In one aspect, the screening method incorporates oral care compositions comprising a metal salt (e.g., stannous fluoride), and uses the resulting biofilm from the treatment with the metal salt as a positive control to select a candidate oral care composition for further development.

In one aspect, the invention contemplates a first Method (1.0), a method of detecting in situ derived biofilm, wherein the method comprises:
a. Administering an intraoral appliance to a subject, wherein the subject wears the intraoral appliance in the subject's oral cavity, and wherein at least a portion of the oral appliance comprises an attachment surface for the biofilm (e.g., hydroxyapatite (HAP));
b. wearing the intraoral appliance (e.g., for at least 24 hours);
c. obtaining a control sample of biofilm present on the attachment surface of the oral appliance, wherein the control sample is treated with an oral care composition that does not contain one or more metal salt(s) (e.g., the control sample does not contain stannous fluoride), and wherein the biofilm is treated with the oral care composition while it is in the oral cavity;
d. obtaining a test sample of biofilm present on the attachment surface of the oral appliance, wherein the tested sample is treated with an oral care composition comprising a metal salt (e.g., stannous fluoride and/or zinc phosphate) while the biofilm is in the oral cavity;
e. labeling the control sample of biofilm, and the test sample of biofilm, with one or more microbial fluorescent probe(s);
f. imaging labeled cells on the biofilms by measuring fluorescence light emitted from the microbial labeled cells by confocal laser scanning microscopy (CLSM);
g. quantifying surface property changes on the biofilms by atomic force microscopy (AFM), wherein the changes in property of the test sample are relative to the control sample;
h. determining the architectural changes in the test sample of biofilm relative to the control sample of biofilm; and
i. detecting or measuring the type (e.g., bacterial species) and/or abundance (e.g., numbers of bacterial colonies) of in situ biofilm present in the test sample of biofilm, and comparing it to the control sample of biofilm.

Method 1.0 also encompasses the following aspects:
1.1 Method of 1.0, wherein the attachment surface can be selected from: human enamel, bovine enamel, bovine dentine, hydroxyapatite, polished glass, and titanium
1.2 Method of 1.1, wherein the attachment surface is Hydroxylapatite (HAP) and the HAP surface is smooth and does not contain any grooves;
1.3 Any of the preceding methods, wherein the metal salt is selected from a zinc salt, stannous salt, a copper salt, and combinations thereof.
1.4 The Method of 1.3, wherein the metal salt is a zinc salt comprises one or more salts selected from the group consisting of: zinc citrate, zinc oxide, zinc chloride, zinc lactate, zinc nitrate, zinc acetate, zinc gluconate, zinc glycinate, zinc sulfate, zinc phosphate and combinations thereof.
1.5 The Method of 1.3 or 1.4, wherein the zinc salt comprises zinc citrate and zinc oxide.
1.6 The Method of 1.3 or 1.4, wherein the zinc salt comprises zinc phosphate.
1.7 The Method of 1.3, wherein the metal is a stannous salt comprises one or more salts selected from the group consisting of: stannous fluoride, stannous pyrophosphate, and combinations thereof.
1.8 The Method of 1.3 or 1.7, wherein the stannous salt is stannous fluoride.
1.9 The Method of 1.8, wherein the amount of stannous fluoride is from 0.1-2.0% by wt. % (e.g., 0.454% by wt.) of the total oral care composition.
1.10 The method of any of the preceding methods, wherein the metal salt comprises both stannous fluoride and zinc phosphate.
1.11 Any of the preceding methods, wherein the subject brushes their teeth from 1-3 times a day (e.g., 2 times/day).
1.12 Any of the preceding methods, wherein the intraoral device is worn at all times, except during oral hygiene and while eating and drinking.
1.13 Any of the preceding methods, wherein the test biofilm is treated with the metal salt while it is attached to the HAP disk in the oral cavity of the subject.
1.14 Any of the preceding methods, wherein the by confocal laser scanning microscopy or atomic force microscopy is used to visualize architectural changes in the biofilm.
1.15 Method of 1.14, wherein the architectural changes in biofilm can be one or more selected from any of the following: pattern of biofilm formation and growth, assembly of individual microcolonies, dynamics of microbial population growth, cell viability, the number of live or viable bacteria with intact membranes within the colony, visualization of the spatial structure of the microcolonies, architecture of the microcolonies (e.g., which may include characterizing the biofilm, volume, height of each microcolony), three-dimensional structure of microcolonies and biofilm, architecture of each microbial community, spatio-temporal distribution of different species of bacteria, visualization of all microbial communities and assembly of microcolonies into a biofilm, biofilm roughness, Stiffness (using Young's modulus) and, Stickiness (Adhesion) of biofilm and/or individual microcolonies to the hard surface or teeth.
1.16 Any of the preceding methods, wherein the method detects and/or measures the presence of one or more biofilm colonies selected from: Actinomyces gerencseriae, Actinomyces israelii, Actinomyces naeslundii, Actinomyces odontolyticus, Actinomyces viscosus, Bacteroides forsythus, Bacteroides gingivalis, Capnocytophaga gingivalis, Campylobacter gracilis, Campylobacter rectus, Capnocytophaga ochraceu, Capnocytophaga sputigena, Eikenella corrodens, Eubacterium brach, Eubacterium lentum, Eubacterium nodation, Fusobacterium alocis, Fusobacterium nucleatum ss. fusiforme, Gemella morbillorum, Haemophilus aphrophilus, Lactobacillus uli, Peptostreptococcus micros, Porphyromonas gingivalis, Prevotella intermedia, Prevotella nigrescens, Rothia dentocariosa, Selenomonas flueggeii, Selenomonas noxia, Selenomonas spuhigena, Streptococcus anginosus, Streptococcus crista, Streptococcus gordoniz, Streptococcus oralis, Streptococcus intermedius, Streptococcus mills, Streptococcus mutans, Streptococcus salivarius, Streptococcus sanguis, Treponema denticola and Veillonella parvula.

The method of any of Method 1.0 et seq., wherein one of skill in the art administers an oral care composition (e.g., a toothpaste) to a subject in need thereof, wherein the oral care composition (e.g., toothpaste) comprises a stannous salt (e.g., stannous fluoride) (e.g., stannous fluoride and zinc phosphate), based on the detection of the presence of one or more biofilm colonies detected in situ.

In another aspect, the invention contemplates a second Method (2.0), the invention relates to methods of screening for compounds that promote the growth of beneficial oral bacteria and/or inhibit the growth of pathogenic biofilm, wherein screening steps include:
a. Administering an intraoral appliance to a subject, wherein the subject wears the intraoral appliance in the subject's oral cavity, and wherein at least a portion of the oral appliance comprises an attachment surface for the biofilm (e.g., hydroxyapatite (HA));
b. wearing the intraoral appliance (e.g., for at least 24 hours);
c. obtaining a positive control sample of biofilm present on the attachment surface of the intraoral appliance, wherein the control sample is treated with an oral care composition that comprises one or more metal salt(s) (e.g., stannous fluoride) (e.g., stannous fluoride and zinc phosphate), and wherein the biofilm is treated with the oral care composition while it is in the oral cavity;
d. obtaining a test sample of biofilm present on the attachment surface of the intraoral appliance, wherein the tested sample is treated with an oral care composition comprising a candidate compound while the biofilm is in the oral cavity;
e. labeling the positive control sample of biofilm, and the test sample of biofilm, with one or more microbial fluorescent probe(s);
f. imaging labeled cells on the biofilms by measuring fluorescence light emitted from the microbial labeled cells by confocal laser scanning microscopy (CLSM);
g. quantifying surface property changes on the biofilms by atomic force microscopy (AFM);
h. determining the architectural changes in the test sample of biofilm relative to the control sample of biofilm; and
i. detecting or measuring the type and/or abundance of in situ biofilm present in the test sample of biofilm, and comparing it to the control sample of biofilm.
j. selecting a candidate compound for further development based on its ability to promote the growth of beneficial oral bacteria and/or inhibit the growth of pathogenic biofilm relative to the positive control treated biofilm.

In another aspect, Method 2.0 also encompasses the following aspects:
2.1 Method of 2.0, wherein the attachment surface can be selected from: human enamel, bovine enamel, bovine dentine, hydroxyapatite, polished glass, and titanium
2.2 Method of 2.1, wherein the attachments surface is hydroxyapatite (HA), and wherein the surface is smooth and does not contain any grooves;
2.3 Any of the preceding methods, wherein the metal salt is selected from a zinc salt, stannous salt, a copper salt, and combinations thereof.
2.4 The Method of 2.4, wherein the metal salt is a zinc salt comprises one or more salts selected from the group consisting of: zinc citrate, zinc oxide, zinc chloride, zinc lactate, zinc nitrate, zinc acetate, zinc gluconate, zinc glycinate, zinc sulfate, zinc phosphate and combinations thereof.
2.5 The Method of 2.3 or 2.4, wherein the zinc salt comprises zinc citrate and zinc oxide.
2.6 The Method of 2.3 or 2.4, wherein the zinc salt comprises zinc phosphate.
2.7 The Method of 2.3, wherein the metal is a stannous salt comprises one or more salts selected from the group consisting of: stannous fluoride, stannous pyrophosphate, and combinations thereof.
2.8 The Method of 2.3 or 2.7, wherein the stannous salt is stannous fluoride.
2.9 The Method of 2.8, wherein the amount of stannous fluoride is from 0.1-2.0% by wt. % (e.g., 0.454% by wt.) of the total oral care composition.
2.10 Any of the preceding methods, where the metal salt comprises zinc phosphate and stannous fluoride.
2.11 Any of the preceding methods, wherein the subject brushes their teeth from 1-3 times a day (e.g., 2 times/day).
2.12 Any of the preceding methods, wherein the intraoral device is worn at all times, except during oral hygiene and while eating and drinking.
2.13 Any of the preceding methods, wherein the test biofilm is treated with the metal salt while it is attached to the HAP disk in the oral cavity of the subject.
2.14 Any of the preceding methods, wherein the by confocal laser scanning microscopy or atomic force microscopy is used to visualize architectural changes in the biofilm.
2.15 Method of 2.14, wherein the architectural changes in biofilm can be one or more selected from any of the following: biofilm formation, bacterial colonization, pattern of biofilm formation and biofilm growth, assembly of individual microcolonies, dynamics of microbial population growth, cell viability, the number of live or viable bacteria with intact membranes within the colony, visualization of the spatial structure of the microcolonies, (e.g., which may include characterizing architecture of the microcolonies and the biofilm, volume, height of each microcolony), three-dimensional structure of microcolonies and biofilm, architecture of each microbial community, spatio-temporal distribution of different species of bacteria, visualization of all microbial communities and assembly of microcolonies into a biofilm, biofilm roughness, Stiffness (using Young's modulus) and, Stickiness (Adhesion) of biofilm or microcolonies to the hard surface or teeth.
2.16 Any of the preceding methods, wherein the method detects and/or measures the presence of one or more biofilm colonies selected from: Actinomyces gerencseriae, Actinomyces israelii, Actinomyces naeslundli, Actinomyces odontolyticus, Actinomyces viscosus, Bacteroides forsythus, Bacteroides gingivalis, Capnocytophaga gingivalis, Campylobacter gracilis, Campylobacter rectus, Capnocytophaga ochraceu, Capnocytophaga sputigena, Eikenella corrodens, Eubacterium brach, Eubacterium lentum, Eubacterium nodation, Fusobacterium alocis, Fusobacterium nucleatum ss. fusiforme, Gemella morbillorum, Haemophilus aphrophilus, Lactobacillus uli, Peptostreptococcus micros, Porphyromonas gingivalis, Prevotella intermedia, Prevotella nigrescens, Rothia dentocariosa, Selenomonas flueggeii, Selenomonas noxia, Selenomonas spuhigena, Streptococcus anginosus, Streptococcus crista, Streptococcus gordoniz, Streptococcus oralis, Streptococcus intermedius, Streptococcus mills, Streptococcus mutans, Streptococcus salivarius, Streptococcus sanguis, Treponema denticola and Veillonella parvula.
2.17 Any of the preceding methods, wherein the candidate compound is selected for further development and incorporated within a dentifrice (e.g., toothpaste), gel, lozenge, mint, chewing gum or other suitable oral care formulation.
2.18 The method of any of the preceding methods, wherein the candidate compound is incorporated within a dentifrice (e.g., toothpaste) gel, lozenge, mint, chewing gum or other suitable oral care formulation and administered to a subject in need thereof.

Further provided is the use of a candidate compound selected from Method 2.0, et seq, for prophylaxis or reduction of tooth decay, caries and/or gum disease, or to enhance the growth of beneficial bacteria in the oral cavity, dental caries, gingivitis. periodontitis. inflammation, and gum disease, e.g., by contacting the dental surface with a candidate compound of Method 2.0, et seq, to a patient in need thereof.

In still another aspect, the invention relates to the use of a candidate compound selected from Method 2.0, et seq, in the manufacture of an oral care product to promote growth of beneficial indigenous (endogenous) bacteria, but not the growth of harmful bacteria.

Further provided is use, a candidate compound selected from Method 2.0, et seq, to:
(a) selectively promote growth, metabolic activity or colonization of bacteria that have beneficial effects on oral health, relative to growth, metabolic activity or colonization of pathogenic oral bacteria; or
(b) selectively promote biofilm formation by bacteria that have beneficial effects on oral health, relative to biofilm formation by pathogenic oral bacteria; or
(c) maintain and/or re-establish a healthy oral microbiota in a subject; or (d) treat or prevent one or more of gingivitis, periodontitis, peri-implantitis, peri-implant mucositis, necrotizing gingivitis, necrotizing periodontitis and caries in a subject.

The invention further provides the use of a compound identified in such a screening method of Method 2.0, et seq in any of the herein described methods and uses.

### DETAILED DESCRIPTION

In another aspect, the biofilm collected in any of Method 1.0 et seq, or Method 2.0 et seq, can be tested to determine rheology, tribology, uniaxial compression, and compression force. “Rheology” in this context refers to the product of calculated modulus and adhesion. “Tribology” in this context, refers to the product of calculated friction and adhesion. “Uniaxial compression” and “Compression force” are terms that are understood by one of skill in the art, and refer to products of the calculated modulus.

In another aspect, the biofilm collected in any of Method 1.0 et seq, or Method 2.0 et seq, can be tested to determine microscale properties. One of skill in the art will be to understand how to determine these properties. In at least one aspect, these properties can be determined using a microcantilever, flow cell technology, diffusive wave spectroscopy (DWS) (e.g., looking at modulus and viscosity), as well as microindentation.

In another aspect, the biofilm collected in any of Method 1.0 et seq, or Method 2.0 et seq, can be tested to determine nanoscale information. One of skill in the art will understand how to determine these properties. In at least one aspect, these properties can be determined using atomic force microscopy (“AFM”). AFM can be used to determine “Young's Modulus of Elasticity”. Young's modulus of elasticity is, in turn, correlated to stiffness, reversible deformation of biofilm. AFM can also be used to determine nanoindentation—which correlates to hardness, and irreversible deformation. AFM can also be used to determine: “Adhesion” or “Adhesive Forces” (e.g., correlating to the “stickiness” of biofilm, deformation of biofilm, dissipation, and various other friction forces.

The following description of the preferred embodiment(s) is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

In some embodiments, the metal salt referenced herein is added to the oral care composition as a preformed salt. As used herein, the term “preformed salt”—e.g, when used in reference to zinc phosphate—means that the zinc phosphate is not formed in situ in the oral care composition, e.g., through the reaction of phosphoric acid and another zinc salt.

As used herein, the term “biofilm” refers to the layer(s) of cells attached to a surface. A biofilm can include both alive and growing microbe cells as well as dead microbe cells. The biofilm can be composed of one cell type or it may be composed of two or more cell types. Biofilm in a healthy human mouth is a multispecies microbial community containing hundreds of bacterial, viral and fungal species. Microbial diversity in the mouth can be individual-specific and site-specific. A specific type of biofilm is oral plaque biofilm (i.e., biofilm that typically forms on tooth surfaces in the human mouth). Bacteria in a plaque biofilm have significantly different physiological characteristics, e.g. increased resistance to detergents and antibiotics, making biofilm research highly important. The biofilm described herein, are in situ collected biofilm, where the biofilm grows within the oral cavity, and is treated while still in the oral cavity.

As used herein, “surface roughness” refers to the microscopic structural texture of a biofilm surface whereby the nanoscale topographical profiles were generated. Roughness may be measured with a skidded gage, as in methods including, but not limited to, interferometic optical profilometry or stylus profilometry. These methods, have limitations with respect to lateral resolution, height resolution and surface material limitations. In another aspect, biofilm surface roughness can be determined using Atomic Force Microscopy (AFM). AFM is a three-dimensional scanning technique that has <0.2 nm spatial resolution and the ability to measure most types of materials. Surface roughness acquisition via AFM is obtained through the use of a cantilever with a sharp tip at its end that is used to scan the surface. When the tip is brought into proximity of a sample surface, forces between the tip and the sample lead to a deflection of the cantilever according to Hooke's law. One of skill in the art would understand how to measure surface roughness. For example, surface roughness can be measured in terms of a number of parameters known in the art. In some embodiments, roughness is calculated using a parameter selected from: Arithmetic Average Roughness (Ra); Root Mean Square (RMS) roughness (Rq); Maximum Peak Height (Rp); Maximum Valley Depth (Rv); Mean Roughness Depth (Rz); Maximum Roughness Depth (Rt); and Maximum Surface Roughness (Rmax). In some embodiments, surface roughness is measured in terms of average surface roughness (Ra). Ra is the arithmetic average height of roughness component irregularities from the mean line measured within the sampling length. Smaller Ra values indicate smoother surfaces. Surface roughness can be measured by any method known in the art for measuring Ra, such as surface profilometry, surface scanning methods, confocal microscopy, atomic force microscopy, and scanning electron microscopy. Surface roughness can be measured before or after at least one treatment session and prior to any subsequent substantial exposure to other agents, for instance, remineralizing solutions (including saliva), or test agents.

As used herein, terms referring to biofilm: “volume”, “height”, “architecture”, “spatio-temporal” and “3D visualization”, refer to measurements made using confocal laser-scanning microscopy (“CLSM”). CLSM can calculate zoom factor, image geometry, voxel size, scanning speed and averaging are kept identical for the image series in an experiment. For visualization of volume images from CLSM, gamma corrections and background subtractions are applied to reveal the range of staining intensities and remove global background signal, respectively, for each data set. Combined with image processing software, CLSM provides a quantitative assessment of biofilm.

As used herein, “adhesion” refers to the measurement of a particular biofilm's attachment to the surface of a HAP disc.

As used herein, “laser-scanning microscopy” (LSM) and “confocal laser-scanning microscopy” (CLSM) are used interchangeably and refer to the same type of microscopy.

The invention contemplates a number of different surfaces for which the biofilm may attach. For example, Method 1.0 et seq and Method 2.0 et seq, human enamel, bovine enamel, bovine dentine, hydroxyapatite, polished glass, and titanium.

Hydroxyapatite, also called hydroxylapatite, (“HAP”) is a mineral form of calcium apatite generally having the formula Ca₁₀(PO₄)₆(OH)₂. In one particular approach, HAP containing pieces (e.g., small disks) are used. These HAP pieces are relatively small, for example, having an overall volume of 7 mm3 to 110 mm3, preferably from 25 mm3 to 35 mm3.

In one aspect, the in situ plaque biofilm is attached to the surface of a HAP pieces as a result of the HAP pieces being attached to an intraoral appliance (e.g., oral split or mouthpiece) worn by human subjects for a defined period of time. This defined period of time is at least 24 hours for one session. Accordingly, the method may comprise the step of having human subjects wearing the oral appliance for 6 hours to 4 days, e.g., 1-3 days, e.g., 2 days, e.g., 1 day, e.g., 12 hours; wherein at least a portion of the oral appliance comprises HAP as a surface of the biofilm, and wherein the biofilm is an in situ plaque biofilm. As used herein, hydroxylapatite can be referred interchangeably as “HA” or “HAP”.

The term “intraoral appliance” means a device that can be temporarily worn inside the oral cavity (i.e., mouth) of a human subject for up to multiple days at a time (but temporarily removed during eating or oral hygiene and the like). Non-limiting examples of an oral appliance include an oral split, mouthpiece, and retainer. The oral appliance preferably has a plurality of HAP containing pieces (e.g., small disks) releasably attached thereto. In other words, the human subject wears the oral appliance as to allow biofilm to attach/grow to the surfaces of the HAP disk.

The biofilm here is treated with a metal salt, or candidate compound, while in situ. “In situ” as used here, means that which takes place within the organism, specifically within the oral cavity of the human subject. For example, the human subject may wear an oral splint (and the HAP disks releasably attached thereto) while using the stannous containing oral care product. “In situ derived biofilm”, for example, is biofilm that is obtained from the oral cavity of a subject.

The biofilm is labeled with a microbial fluorescent probe. “Microbial fluorescent probe” means a fluorescent probe that binds to microbes of a biofilm. One class such probes includes fluorescently labeled oligonucleotides, preferably rRNA-directed oligonucleotides. Non-limiting examples include SYTO™ branded dyes. One specific example is SYTO-9, wherein excitation is a 485 (DNA) and 486 (RNA), and light emission is detected at 498 (DNA) and 501 (RNA). Within this class of rRNA-directed oligonucleotides dyes, a sub-class of dyes may be used to distinguish between dead or alive microbes. Another class of microbial fluorescent probes include extracellular polymer substances (EPS)-specific fluorescent stains or lectins. A commercially available example of a microbial fluorescent probe is LIVE/DEAD® BacLight™ fluorescence assay stains. These microbial fluorescent probes are widely available as well as the procedure details in how to use them to quantitatively determine the amount of microbes as well as quantitatively determine what portion of these microbes are alive or dead.

### Examples

A healthy subject enrolled in this study is instructed to brush for 2 min, 2 times a day with a washout product, a commercial toothpaste that does not contain stannous fluoride (Control), for the next 14 days. An oral soft tissue assessment is performed on Day 15, a Baseline visit to the dental clinic and the evaluation is recorded as baseline data.

On Baseline visit, Visit 1 the subject receives a personalized, custom-made intraoral appliance that was developed in-house. This is a retainer model designed to mimic the bacterial composition and closely simulate what is observed clinically in a healthy mouth. The subject is instructed to wear the retainer for next 24 hours and continue to brush with the same toothpaste product. The subject is instructed to wear their retainer at all times except during oral hygiene and while eating and drinking. Subject is asked to drop off the retainer after 24 hrs.

On Visit 2 the appliance is submitted for the biofilm analysis. The subject receives a commercially available fluoride toothpaste containing 0.454% stannous fluoride and is instructed to brush for 2 min, 2 times a day with this product) for next 7 days. On Visit 3 the subject receives a new intraoral appliance set and is instructed to wear the retainer for next 24 hours and continue to brush with the stannous fluoride toothpaste product. The subject is instructed to wear their retainer at all times except during oral hygiene and while eating and drinking. Subject is asked to drop off the retainer after 24 hrs.

On Visit 4 the appliance is submitted for the biofilm analysis.

The subject is instructed to continue brushing for 2 min, 2 times a day with the same stannous fluoride toothpaste for the next 7 days.

On Visit 5 the subject receives a new intraoral appliance set and is instructed to wear the retainer for next 24 hours and continue to brush with the stannous fluoride toothpaste product. The subject is instructed to wear their retainer at all times except during oral hygiene and while eating and drinking. Subject is asked to drop off the retainer after 24 hrs.

On Visit 6 the appliance is submitted for the biofilm analysis.

In-Situ Biofilm Model (Retainer with HAP Discs) Preparation and Design

The intraoral appliance/retainer shall be worn by the participants for up to 48 hours, and removed only during brushing, eating, and drinking. The retainer should be placed back into the mouth immediately after brushing and within 30 minutes after eating and drinking.

Atomic Force Microscopy measurements: Measurements are taken by Bruker AFM in air and liquid. In this respect, high-density HAP disks are obtained and samples are prepared by placing the HAP substrate on a magnetic disk and mounting on the J-scanner of the Multimode 8 with a Nanoscope V controller (Bruker). Samples are imaged using PeakForce-Quantitative Nanomechanical Mapping (PeakForce QNM) in air and liquid at a scan rate of 0.5-1.5 Hz. For samples imaged in liquid, a drop of PBS is placed on both the HAP disk and AFM tip. In another aspect

Atomic Force Microscopy Imaging and Data Extraction Parameters: The AFM probe utilized in all imaging analysis is the POINTPROBE-PLUS Silicon-SPM Sensor by Nanosensors. The characteristics of the implored probe include a resonant frequency of 45-115 kHz, a spring constant of 0.5-9.5 N/m, and a nominal radius of <10 nm. For each sample, two to three different areas are tested. Prior to imaging and analysis, the deflection sensitivity is calibrated on a sapphire substrate as well as on PDMS to validate calibration of probes on different materials. Although the spring constant of the probe is known, k is calibrated using the thermal tune method (Lorentzian air) and fit to achieve a value within 10% of the given value. Images were acquired at varying scales between 100 μm and 1 μm, aspect ratio of 1.00, and either 256 or 512 data points/line. Once images are obtained, processing is done using the NanoScope Analysis Software v. 1.5 (Bruker). All plane-fitting and image processing is consistent through all images analyzed (Plane-Fit and 2nd order flatten) for obtaining roughness parameters.

Force curves are taken per pixel and fit using Nanoscope Analysis Software v. 1.5, whereby the baseline for each force curve was corrected and the retract curve (which shows both peak force (maximum force) and adhesion force (minimum force) is fit using the Hertzian method. The Young's modulus values were extracted using the Hertzian model for a spherical indenter, where force is related to the indentation depth from three equations. The Hertz model is used as it has been extensively applied to biofilm mechanical analysis using AFM2-4.

Confocal Laser-Scanning Microscopy measurements: Serial images of fluorescent signals are recorded by confocal laser-scanning microscopy with a Nikon imaging system using a 63× oil lens magnification. Syto 9 and PI signals are acquired sequentially from each sample at five randomly selected positions: centre, right, left, top and bottom. A series of optical sections is scanned at specific depths, and then each section of 1024×1024 pixels is “stacked” using the Nikon imaging software. This gives rise to either a two-dimensional image that includes all planes of focus in the sample or a computer-generated three-dimensional image. This gives unprecedented resolution in viewing oral biofilm samples, enabling individual bacteria or smaller colonies to be better differentiated from larger colonies, as well as giving insight into the three-dimensional spatial relationships of microbial communities in their environment. The volumes are sampled according to the Nyquist rate (2× oversampling). Zoom factor, image geometry, voxel size, scanning speed and averaging are kept identical for the image series in an experiment. For visualization of volume images, gamma corrections and background subtractions are applied to reveal the range of staining intensities and remove global background signal, respectively, for each data set. Combined with image processing software, CLSM provides a quantitative assessment of biofilm.

The biofilm structure is quantified using Imaris 3D image processing software (Version 8.4, Bitplane, Oxford instruments, Zurich, Switzerland). It is used to convert pixels from confocal image stacks into numerical values, facilitating quantitative characterization of each structural component within 3D biofilm images. Imaris software allows the determination of mean biofilm thickness for all tested conditions. The average thickness is calculated from the base at the biofilm—HAP interface to the top of the biofilm in both channels, across the entire biofilm in the field of view. Mean biofilm thickness provides a measure of the spatial size of the biofilm. Fluorescent signals are reported as the intensity sum of voxels per channel in 3D-reconstructed images, using manually defined 3D surfaces around the biofilm using the Surface tool from Imaris software.

Upon collection of samples, and analysis of data, significant alterations in patterns of biofilm formation and changes in architectural shifts and biophysical properties are revealed in in-situ grown oral biofilm when post brushing with toothpaste containing Sn for 7 or 14 days. As noted above, the effect of stannous is analyzed by Qualitative and quantitative approaches using two imaging techniques, LSM (laser scanning microscopy) and AFM (atomic force microscopy). The results demonstrates that upon continuous brushing with stannous fluoride (an active) toothpaste the biofilm microcolonies as well as their distribution becomes smaller and there is a reduction in stickiness (adhesion or adhesive forces) and an increase in stiffness of these bacterial colonies.

For example, by using LSM, the biofilm thickness of stannous treated samples, and control, are tested over the course of the study. Measurements of the “Control” are taken after 14 days of brushing with a product that does not contain stannous fluoride. Measurements of the “Stannous Fluoride Sample” are taken at after seven days (“Week 1”) and fourteen days (“Week 2”) following brushing with a product containing stannous fluoride. The results are described in Table 1 below:

TABLE 1
Biofilm Thickness (microns)

| | | Stannous Fluoride Sample | |
|---|---|---|---|
| Measurement | Control | Week 1 | Week 2 |
| 1 | 34.0 | 7.6 | 7.4 |
| 2 | 38.1 | 9.0 | 7.6 |
| 3 | 22.0 | 4.0 | 5.3 |
| 4 | 21.1 | 6.5 | 8.5 |
| 5 | 26.9 | 5.5 | 10.6 |
| 6 | 20.2 | 6.0 | 10.8 |
| 7 | 21.7 | 7.0 | 9.9 |
| 8 | 26.6 | 7.0 | 12.4 |

LSM is also used to measure biofilm volume and voxels over the course of the study. In this context, “voxels” are representative of the number of bacterial colonies are present in the analyzed sample. Measurements of the “Control” are taken after 14 days of brushing with a product that does not contain stannous fluoride. Measurements of the “Stannous Fluoride Sample” are taken at after seven days (“Week 1”) and fourteen days (“Week 2”) following brushing with a product containing stannous fluoride. The results are described in Table 2 and Table 3 below:

TABLE 2
Volume (μm³)

| | | Stannous Fluoride Sample | |
|---|---|---|---|
| Measurement | Control | Week 1 | Week 2 |
| 1 | 552391 | 13585 | 2945 |
| 2 | 484880 | 45899 | 1665 |
| 3 | 494890 | 7549 | 554 |
| 4 | 466561 | 8239 | 500 |

TABLE 3
Voxel Count

| | | Stannous Fluoride Sample | |
|---|---|---|---|
| Measurement | Control | Week 1 | Week 2 |
| 1 | 25700000 | 315375 | 68796 |
| 2 | 22700000 | 5340000 | 77568 |
| 3 | 23100000 | 877952 | 25740 |
| 4 | 21700000 | 961947 | 23166 |

For example, from the topography of the samples, the roughness of the HAP surface decreases after biofilm growth and increases after the panelist brushed with a toothpaste containing stannous fluoride for 14 days. As seen in Table 4, which measures “Roughness”, the roughness of the HAP surface after one or two weeks of brushing with the stannous fluoride active is comparable to the control HAP surface:

TABLE 4
Measurement of HAP surface “Roughness” (Nanometers)

| Sample | 15 μm | 25 μm |
|---|---|---|
| HAP Surface (Control) | 141 | 231.2 |
| HAP Surface + Biofilm | 36.1 | 67.3 |
| Week 1 (Use of Toothpaste | 60.6 | 231 |
| containing Stannous Fluoride) | | |
| Week 2 (Use of Toothpaste | 104.9 | 220 |
| containing Stannous Fluoride) | | |

TABLE 5
Measurement of Biofilm “Stiffness” and
“Adhesion” to the HAP Surface

| Sample | Stiffness (GPa) | Adhesion (nN) |
|---|---|---|
| HAP Surface (Control) | 7.4 | 25.6 |
| HAP Surface + Biofilm | 0.5 | 59.2 |
| Week 1 (Use of Toothpaste | 5.5 | 35.4 |
| containing Stannous Fluoride) | | |
| Week 2 (Use of Toothpaste | 7.3 | 17.2 |
| containing Stannous Fluoride) | | |

From FIG. 3 above, “Stiffness” is measured by the Young's modulus, which decreases after biofilm growth, and increases after the panelist brushed with a toothpaste containing a stannous fluoride salt, for 14 days. This implies that the surface is becomes softer. The adhesive force between surface and AFM tip increased after biofilm growth and decreased after the panelist brushed with Colgate Total for 14 days implying that biofilm renders the HAP surface stickier. Upon brushing for 14 days with Colgate Total, the adhesive forces decrease. These results render unique data about the effects of the stannous fluoride toothpaste, and provides unique information about the characteristics of the biofilm that forms in situ during these trials, and which is exposed to the stannous active while in the oral cavity of the human subject.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of detecting in situ derived biofilm, wherein the method comprises:
a. Administering an intraoral appliance to a subject, wherein the subject wears the intraoral appliance in the subject's oral cavity, and wherein at least a portion of the oral appliance comprises an attachment surface for the biofilm;
b. wearing the intraoral appliance for at least 24 hours; obtaining a control sample of biofilm present on the attachment surface of the oral appliance, wherein the control sample is treated with an oral care composition that does not contain one or more metal salt(s), and wherein the biofilm is treated with the oral care composition while it is in the oral cavity;
c. obtaining a test sample of biofilm present on the attachment surface of the oral appliance, wherein the tested sample is treated with an oral care composition comprising a metal salt while the biofilm is in the oral cavity;
d. labeling the control sample of biofilm, and the test sample of biofilm, with one or more microbial fluorescent probe(s);
e. imaging labeled cells on the biofilms by measuring fluorescence light emitted from the microbial labeled cells by confocal laser scanning microscopy (CLSM);
f. quantifying surface property changes on the biofilms by atomic force microscopy (AFM), wherein the changes in property of the test sample are relative to the control sample;
g. determining the architectural changes in the test sample of biofilm relative to the control sample of biofilm; and
h. detecting or measuring the type and/or abundance of in situ biofilm present in the test sample of biofilm, and comparing it to the control sample of biofilm.

2. The method of claim 1, wherein the attachment surface can be selected from: human enamel, bovine enamel, bovine dentine, hydroxyapatite, polished glass, and titanium

3. The method of claim 1, wherein the metal salt is selected from a zinc salt, stannous salt, a copper salt, and combinations thereof.

4. The method of claim 3, wherein the metal salt is a zinc salt comprises one or more salts selected from the group consisting of: zinc citrate, zinc oxide, zinc chloride, zinc lactate, zinc nitrate, zinc acetate, zinc gluconate, zinc glycinate, zinc sulfate, zinc phosphate and combinations thereof.

5. The method of claim 3, wherein the zinc salt comprises zinc citrate and zinc oxide.

6. The method of claim 3, wherein the zinc salt comprises zinc phosphate.

7. The method of claim 3, wherein the metal is a stannous salt comprises one or more salts selected from the group consisting of: stannous fluoride, stannous pyrophosphate, and combinations thereof.

8. The method of claim 3, wherein the stannous salt is stannous fluoride.

9. (canceled)

10. The method of claim 1, wherein the by confocal laser scanning microscopy or atomic force microscopy is used to visualize architectural changes in the biofilm, and wherein the architectural changes in biofilm can be one or more selected from any of the following: biofilm formation, bacterial colonization, pattern of biofilm formation and biofilm growth, assembly of individual microcolonies, dynamics of microbial population growth, cell viability, the number of live or viable bacteria with intact membranes within the colony, visualization of the spatial structure of the microcolonies, architecture of the microcolonies and the biofilm, volume, height of each microcolony, 3D structure of microcolonies and biofilm, architecture of each microbial community, spatio-temporal distribution of different species of bacteria, visualization of all microbial communities and assembly of microcolonies into a biofilm, biofilm roughness, Stiffness (using Young's modulus) and, Stickiness (Adhesion) of biofilm, biofilm formation, viability, volume, height, architecture, spatio-temporal and 3D visualization, biofilm roughness, Stiffness (using Young's modulus) and, Stickiness (Adhesion or Adhesive Forces) of biofilm growth.

11. The method of claim 1, wherein the method detects and/or measures the presence of one or more biofilm colonies selected from: Actinomyces gerencseriae, Actinomyces israelii, Actinomyces naeslundli, Actinomyces odontolyticus, Actinomyces viscosus, Bacteroides forsythus, Bacteroides gingivalis, Capnocytophaga gingivalis, Campylobacter gracilis, Campylobacter rectus, Capnocytophaga ochraceu, Capnocytophaga sputigena, Eikenella corrodens, Eubacterium brach, Eubacterium lentum, Eubacterium nodation, Fusobacterium alocis, Fusobacterium nucleatum ss. fusiforme, Gemella morbillorum, Haemophilus aphrophilus, Lactobacillus uli, Peptostreptococcus micros, Porphyromonas gingivalis, Prevotella intermedia, Prevotella nigrescens, Rothia dentocariosa, Selenomonas flueggeii, Selenomonas noxia, Selenomonas spuhigena, Streptococcus anginosus, Streptococcus crista, Streptococcus gordoniz, Streptococcus oralis, Streptococcus intermedius, Streptococcus mills, Streptococcus mutans, Streptococcus salivarius, Streptococcus sanguis, Treponema denticola and Veillonella parvula.

12. A method of screening for compounds that promote the growth of beneficial oral bacteria and/or inhibit the growth of pathogenic biofilm, wherein the screening steps include:
a. Administering an intraoral appliance to a subject, wherein the subject wears the intraoral appliance in the subject's oral cavity, and wherein at least a portion of the oral appliance comprises an attachment surface for the biofilm (e.g., hydroxyapatite (HA));
b. wearing the intraoral appliance for at least 24 hours;
c. obtaining a positive control sample of biofilm present on the attachment surface of the intraoral appliance, wherein the control sample is treated with an oral care composition that contains one or more metal salt(s), and wherein the biofilm is treated with the oral care composition while it is in the oral cavity;
d. obtaining a test sample of biofilm present on the attachment surface of the intraoral appliance, wherein the tested sample is treated with an oral care composition comprising a candidate compound while the biofilm is in the oral cavity;
e. labeling the positive control sample of biofilm, and the test sample of biofilm, with one or more microbial fluorescent probe(s);
f. imaging labeled cells on the biofilms by measuring fluorescence light emitted from the microbial labeled cells by confocal laser scanning microscopy (CLSM);
g. quantifying surface property changes on the biofilms by atomic force microscopy (AFM);
h. determining the architectural changes in the test sample of biofilm relative to the control sample of biofilm; and
i. detecting or measuring the type and/or abundance of in situ biofilm present in the test sample of biofilm, and comparing it to the control sample of biofilm.
j. selecting a candidate compound for further development based on its ability to promote the growth of beneficial oral bacteria and/or inhibit the growth of pathogenic biofilm relative to the positive control treated biofilm.

13. The method of claim 12, wherein the attachment surface can be selected from: human enamel, bovine enamel, bovine dentine, hydroxyapatite, polished glass, and titanium

14. The method of claim 12, wherein the metal salt is selected from a zinc salt, stannous salt, a copper salt, and combinations thereof.

15. The method of claim 14, wherein the metal salt is a zinc salt comprises one or more salts selected from the group consisting of: zinc citrate, zinc oxide, zinc chloride, zinc lactate, zinc nitrate, zinc acetate, zinc gluconate, zinc glycinate, zinc sulfate, zinc phosphate and combinations thereof.

16. The method of claim 14, wherein the zinc salt comprises zinc citrate and zinc oxide.

17. The method of claim 14, wherein the zinc salt comprises zinc phosphate.

18. The method of claim 14, wherein the metal is a stannous salt comprises one or more salts selected from the group consisting of: stannous fluoride, stannous pyrophosphate, and combinations thereof.

19. The method of claim 14, wherein the stannous salt is stannous fluoride.

20. The method of claim 12, wherein the by confocal laser scanning microscopy or atomic force microscopy is used to visualize architectural changes in the biofilm, and wherein the architectural changes in biofilm can be one or more selected from any of the following: biofilm formation, bacterial colonization, pattern of biofilm formation and biofilm growth, assembly of individual microcolonies, dynamics of microbial population growth, cell viability, the number of live or viable bacteria with intact membranes within the colony, visualization of the spatial structure of the microcolonies, architecture of the microcolonies and the biofilm, volume, height of each microcolony, 3D structure of microcolonies and biofilm, architecture of each microbial community, spatio-temporal distribution of different species of bacteria, visualization of all microbial communities and assembly of microcolonies into a biofilm, biofilm roughness, Stiffness (using Young's modulus) and, Stickiness (Adhesion or Adhesive Forces) of biofilm. biofilm formation, viability, volume, height, architecture, spatio-temporal and 3D visualization, biofilm roughness, Stiffness (using Young's modulus) and, Stickiness (Adhesion) of biofilm growth.

21. The method of claim 12, wherein the method detects and/or measures the presence of one or more biofilm colonies selected from: Actinomyces gerencseriae, Actinomyces israelii, Actinomyces naeslundli, Actinomyces odontolyticus, Actinomyces viscosus, Bacteroides forsythus, Bacteroides gingivalis, Capnocytophaga gingivalis, Campylobacter gracilis, Campylobacter rectus, Capnocytophaga ochraceu, Capnocytophaga sputigena, Eikenella corrodens, Eubacterium brach, Eubacterium lentum, Eubacterium nodation, Fusobacterium alocis, Fusobacterium nucleatum ss. fusiforme, Gemella morbillorum, Haemophilus aphrophilus, Lactobacillus uli, Peptostreptococcus micros, Porphyromonas gingivalis, Prevotella intermedia, Prevotella nigrescens, Rothia dentocariosa, Selenomonas flueggeii, Selenomonas noxia, Selenomonas spuhigena, Streptococcus anginosus, Streptococcus crista, Streptococcus gordoniz, Streptococcus oralis, Streptococcus intermedius, Streptococcus mills, Streptococcus mutans, Streptococcus salivarius, Streptococcus sanguis, Treponema denticola and Veillonella parvula.
